(19) United States
(12) Patent Application Publication
(10) Pub. No.: US 2006/0281800 A1
(43) Pub. Date: Dec. 14, 2006

(54) Polymorphic form of olmesartan and process for its preparation

(75) Inventors: Bobba Venkata Siva Kumar, Koper Khairne (IN); Girish Bansilal Patel, Airoli (IN); Nitin Sharad Chandra Pradhan, Thane (W) (IN); Shekhar Bhaskar Bhirud, Vashi (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/402,738

(22) Filed: Apr. 12, 2006

Related U.S. Application Data
(60) Provisional application No. 60/677,578, filed on May 4, 2005.

(30) Foreign Application Priority Data
Apr. 12, 2005 (IN) 461/MUM/2005

(51) Int. Cl. A61K 31/4178; C07D 405/14

(52) U.S. Cl. 514/381; 548/253

(57) ABSTRACT

A process for the preparation of a novel crystalline polymorph of olmesartan medoxomil, designated Form G, is provided comprising the steps of (a) preparing a solution comprising olmesartan medoxomil and one or more solvents selected from the group consisting of a nitrile, alcohol and mixtures thereof at a suitable temperature to obtain a solution; and (b) recovering olmesartan medoxomil substantially in polymorph Form G from the solution. A novel polymorph Form G of olmesartan medoxomil and pharmaceutical compositions containing same are also provided herein.

### PRIORITY

This application claims the benefit under 35 U.S.C. §119 to U.S. Provisional Application No. 60/677,578, filed on May 4, 2005, and entitled “POLYMORPHIC FORM OF OLMESARTAN AND PROCESS FOR ITS PREPARATION” and to Indian Provisional Application 461/MUM/2005, filed on Apr. 12, 2005, and entitled “POLYMORPHIC FORM OF OLMESARTAN AND PROCESS FOR ITS PREPARATION”, the contents of each of which are incorporated by reference herein.

### BACKGROUND OF THE INVENTION

1. Technical Field

The present invention generally relates to a novel polymorphic form of olmesartan medoxomil and a process for its preparation.

2. Description of the Related Art

The chemical name for olmesartan medoxomil is 2,3-dihydroxy-2-butenyl 4-(1-hydroxy-1-methylethyl)-2-propyl-1-[p-(o-1H-tetrazol-5-ylphenyl)benzyl]imidzole-5- carboxylate, cyclic 2,3-carbonate. Olmesartan medoxomil is represented by the structure of formula I.

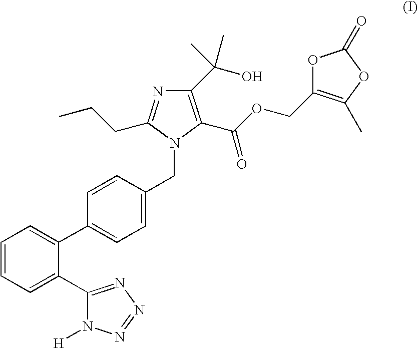

Olmesartan medoxomil is a prodrug that is hydrolyzed to olmesartan during absorption from the gastrointestinal tract. Olmesartan is a selective AT₁ subtype angiotensin II receptor antagonist. Angiotensin II is formed from angiotensin I in a reaction catalyzed by angiotensin converting enzyme (ACE, kinase II). Angiotensin II is the principal pressor agent of the renin-angiotensin system, with effects that include vasoconstriction, stimulation of synthesis and release of aldosterone, cardiac stimulation and renal reabsorption of sodium. Olmesartan blocks the vasoconstrictor effects of angiotensin II by selectively blocking the binding of angiotensin II to the AT1 receptor in vascular smooth muscle. Olmesartan medoxomil is indicated for hypertension and is commercially sold under the trade name Benicar®. See, e.g., The Merck Index, Thirteenth Edition, 2001, pp. 1223-24, monograph 6909; and Physician's Desk Reference, “Benicar,” 58^{th} Edition, pp. 3000-3001 (2004).

U.S. Pat. No. 5,616,599 (“the '599 patent”), herein incorporated by reference, discloses olmesartan medoxomil and a process for its preparation.

Polymorphism is the occurrence of different crystalline forms of a single compound and it is a property of some compounds and complexes. Thus, polymorphs are distinct solids sharing the same molecular formula, yet each polymorph may have distinct physical properties. Therefore, a single compound may give rise to a variety of polymorphic forms where each form has different and distinct physical properties, such as different solubility profiles, different melting point temperatures and/or different x-ray diffraction peaks. Since the solubility of each polymorph may vary, identifying the existence of pharmaceutical polymorphs is essential for providing pharmaceuticals with predicable solubility profiles. It is desirable to investigate all solid state forms of a drug, including all polymorphic forms, and to determine the stability, dissolution and flow properties of each polymorphic form. Polymorphic forms of a compound can be distinguished in a laboratory by X-ray diffraction spectroscopy and by other methods such as, infrared spectrometry. Additionally, polymorphic forms of the same drug substance or active pharmaceutical ingredient, can be administered by itself or formulated as a drug product (also known as the final or finished dosage form), and are well known in the pharmaceutical art to affect, for example, the solubility, stability, flowability, tractability and compressibility of drug substances and the safety and efficacy of drug products.

The discovery of new polymorphic forms of a pharmaceutically useful compound provides a new opportunity to improve the performance characteristics of a pharmaceutical product. It also adds to the material that a formulation scientist has available for designing, for example, a pharmaceutical dosage form of a drug with a targeted release profile or other desired characteristic. It has now been surprisingly found that a new crystalline form of olmesartan medoxomil exists and a process for its preparation.

### SUMMARY OF THE INVENTION

In accordance with one embodiment of the present invention, a process for the preparation of olmesartan medoxomil substantially in polymorph Form G is provided comprising the steps of:
(a) preparing a solution comprising olmesartan medoxomil and one or more solvents selected from the group consisting of a nitrile, alcohol and mixtures thereof at a suitable temperature; and
(b) recovering olmesartan medoxomil substantially in polymorph Form G from the solution.

In accordance with a second embodiment of the present invention, olmesartan medoxomil substantially in the polymorph Form G is provided.

In accordance with a third embodiment of the present invention, olmesartan medoxomil substantially in the polymorph Form G and exhibiting a characteristic peak (expressed in degrees 2θ±0.2°θ) at about 13.4 and 23.1 is provided.

In accordance with a fourth embodiment of the present invention, olmesartan medoxomil substantially in the polymorph Form G and exhibiting characteristic peaks (expressed in degrees 2θ±0.2°θ) at approximately one or more of the positions: about 13.4, 16.8, 17.7 and/or 23.1 is provided.

In accordance with a fifth embodiment of the present invention, olmesartan medoxomil substantially in polymorph Form G and characterized by having at least one of the following properties is provided: (a) a differential scanning calorimetric (DSC) thermogram substantially in accordance with FIG. 1, (b) a X-ray diffraction (XRD) pattern substantially in accordance with FIG. 2, and/or (C) a Thermogavimetric Analysis (TGA) substantially in accordance with FIG. 3.

In accordance with a sixth embodiment of the present invention, olmesartan medoxomil substantially in the polymorph Form G prepared by the process comprising (a) preparing a solution comprising olmesartan medoxomil and one or more solvents selected from the group consisting of a nitrile, alcohol and mixtures thereof at a suitable temperature; and (b) recovering olmesartan medoxomil in polymorph Form G from the solution is provided.

In accordance with a seventh embodiment of the present invention, a pharmaceutical composition is provided comprising a therapeutically effective amount of olmesartan medoxomil substantially in the polymorph Form G and at least one pharmaceutically acceptable excipient.

### DEFINITIONS

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host means causing a therapeutically effective blood concentration of the active ingredient at the particular location. This can be accomplished, e.g., by topical, local or by systemic administration of the active ingredient to the host.

As used herein, the term “buffering agent” is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

As used herein, the term “sweetening agent” is intended to mean a compound used to impart sweetness to a preparation. Such compounds include, by way of example and without limitation, aspartame, dextrose, glycerin, mannitol, saccharin sodium, sorbitol, sucrose, fructose and other such materials known to those of ordinary skill in the art.

As used herein, the term “binders” is intended to mean substances used to cause adhesion of powder particles in tablet granulations. Such compounds include, by way of example and without limitation, acacia alginic acid, tragacanth, carboxymethylcellulose sodium, poly (vinylpyrrolidone), compressible sugar (e.g., NuTab), ethylcellulose, gelatin, liquid glucose, methylcellulose, povidone and pregelatinized starch, combinations thereof and other material known to those of ordinary skill in the art.

When needed, other binders may also be included in the present invention. Exemplary binders include starch, poly(ethylene glycol), guar gum, polysaccharide, bentonites, sugars, invert sugars, poloxamers (PLURONIC™ F68, PLURONIC™ F127), collagen, albumin, celluloses in nonaqueous solvents, combinations thereof and the like. Other binders include, for example, poly(propylene glycol), polyoxyethylene-polypropylene copolymer, polyethylene ester, polyethylene sorbitan ester, poly(ethylene oxide), microcrystalline cellulose, poly(vinylpyrrolidone), combinations thereof and other such materials known to those of ordinary skill in the art.

As used herein, the term “diluent” or “filler” is intended to mean inert substances used as fillers to create the desired bulk, flow properties, and compression characteristics in the preparation of tablets and capsules. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

As used herein, the term “glidant” is intended to mean agents used in tablet and capsule formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

As used herein, the term “lubricant” is intended to mean substances used in tablet formulations to reduce friction during tablet compression. Such compounds include, by way of example and without limitation, calcium stearate, magnesium stearate, mineral oil, stearic acid, zinc stearate, combinations thereof and other such materials known to those of ordinary skill in the art.

As used herein, the term “disintegrant” is intended to mean a compound used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starched thereof, sweeteners, clays, such as bentonite, microcrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

As used herein, the term “wetting agent” is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type, also known as superinone or triton) is another useful wetting agent, combinations thereof and other such materials known to those of ordinary skill in the art.

Most of these excipients are described in detail in, e.g., Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), which are incorporated by reference herein.

### BRIEF DESCRIPTION OF THE DRAWINGS

FIG. 1 is a characteristic DSC thermogram of polymorph Form G of olmesartan medoxomil.

FIG. 2 is a characteristic XRD pattern of polymorph Form G of olmesartan medoxomil.

FIG. 3 illustrates the graphical results of a Thermogavimetric Analysis of polymorph Form G of olmesartan medoxomil.

### DETAILED DESCRIPTION OF THE PREFERRED EMBODIMENTS

The present invention is directed to a novel polymorphic form of olmesartan medoxomil, designated Form G. Olmesartan medoxomil substantially in the polymorph G form may be used in a pharmaceutical composition as, for example, an antihypertensive. In general, a process for preparing olmesartan medoxomil substantially in polymorph Form G includes at least (a) preparing a solution containing at least olmesartan medoxomil with a solvent selected from the group consisting of one or more nitrites, one or more alcohols and mixtures thereof at a suitable temperature; and (b) recovering olmesartan medoxomil substantially in polymorph Form G from the solution.

Olmesartan medoxomil is well known and can be obtained by any method known in the art. See, e.g., U.S. Pat. No. 5,616,599 and U.S. Patent Application Publication No. 2006/0069141, the contents of each of which are incorporated by reference herein. In step (a) of the process of the present invention, a solution containing at least olmesartan medoxomil and one or more solvents selected from the group consisting of a nitrile, alcohol and mixtures thereof is prepared at a suitable temperature by, for example, dissolving olmesartan medoxomil in one or more of the solvents. Suitable nitrites include, but are not limited to, acetonitrile, propionitrile and the like and mixtures thereof. Suitable alcohols include those having from 1 to about 12 carbon atoms such as, for example, methanol, ethanol, isopropyl alcohol, n-butyl alcohol, t-butyl alcohol, and the like and mixtures thereof. Preferred solvents for use herein include acetonitrile, methanol, ethanol and mixtures thereof, and most preferably methanol. The solvent(s) will ordinarily be present in an amount ranging from about 2 volumes to about 30 volumes.

In preparing the solution, olmesartan medoxomil may be stirred in the solvent at a suitable temperature, e.g., a temperature ranging from about 25° C. to about 75° C., preferably at about 30° C. to about 60° C., and most preferably at about 40° C. to about 50° C. The solution may then be cooled to induce crystallization. Preferably, the solution is cooled to a temperature ranging from about -10° C. to about 30° C., preferably to about -5° C. to about 20° C., and most preferably to about 0° C. to about 10° C. by techniques known in the art. A crystalline solid will then precipitate out of the solution. The resulting crystals may then be recovered by techniques well known in the art, e.g., filtration, centrifugation, decanting, etc. The filtered solid may then be washed and dried to produce crystals of Form G of olmesartan medoxomil.

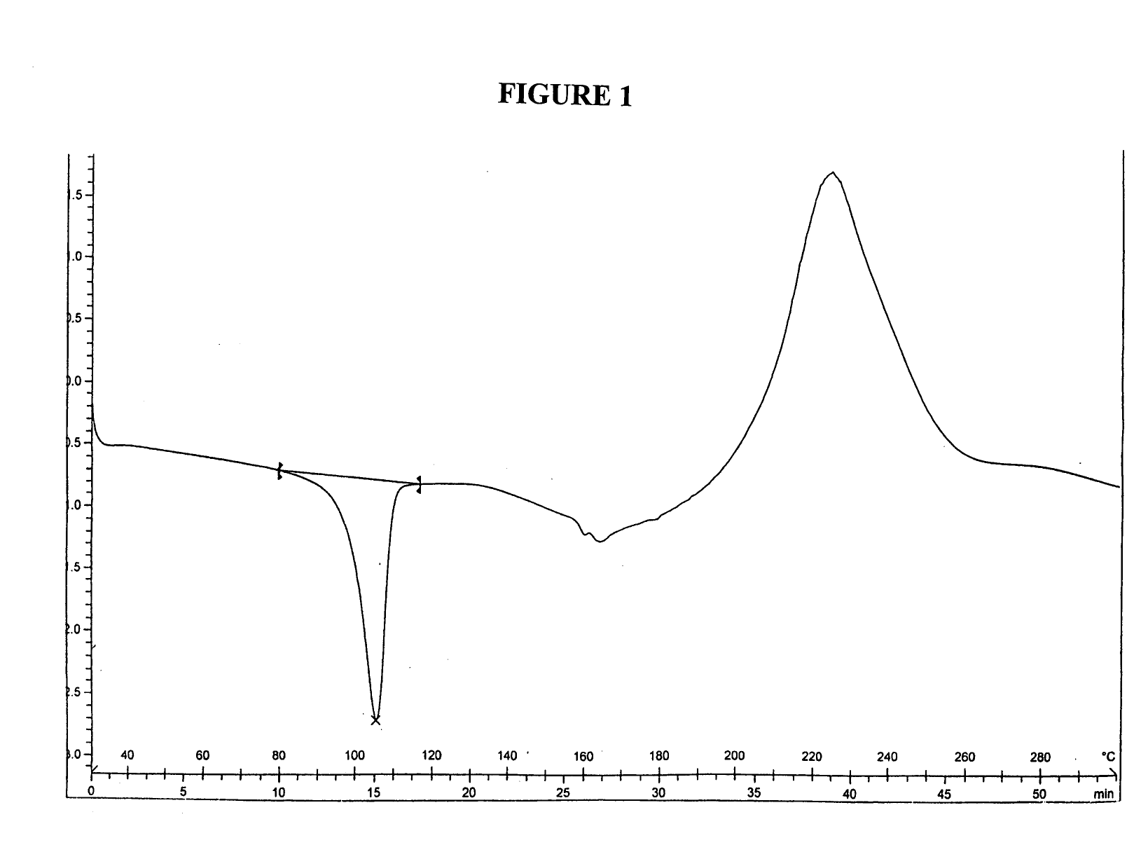

The present invention also provides olmesartan medoxomil substantially in polymorph Form G. Crystallinity of the novel polymorph may be measured using methods familiar to those skilled in the art. The novel polymorph of the present invention as described hereinbelow was characterized by a DSC thermogram, X-ray powder diffraction (XRD), and Thermogavimetric Analysis. As shown in FIG. 1, polymorph Form G of olmesartan medoxomil exhibits a predominant endothermic peak at about 105.23° C. as measured by a Differential Scanning Calorimeter (DSC 822, Mettler Toledo) at a scan rate of 10° C. per minute with an Indium standard. In this regard, it should be understood that the endotherm measured by a particular differential scanning calorimeter is dependent upon a number of factors, including the rate of heating (i.e., scan rate), the calibration standard utilized, instrument calibration, relative humidity, and upon the chemical purity of the sample being tested. Thus, an endotherm as measured by DSC on the instrument identified above may vary by as much as ±1° C. or even ±1½° C. Accordingly, the term “about 105.23° C.” is intended to encompass such instrument variations.

The X-Ray powder diffraction spectrum for the polymorph was measured as known in the art, e.g., by an X-ray powder Diffractometer. In one embodiment, olmesartan medoxomil substantially in polymorph Form G can be characterized by an XRD pattern which exhibits characteristic peaks (expressed in degrees 2θ±0.2°θ) at about 13.4, 16.8, 17.7 and/or 23.1. Additional peaks are listed in Table I below.

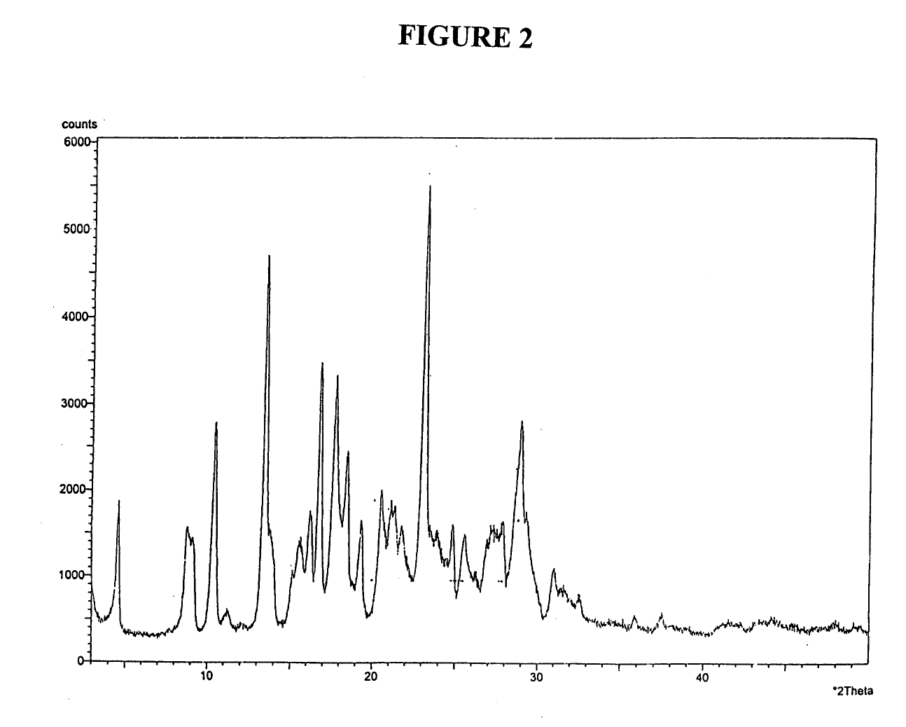

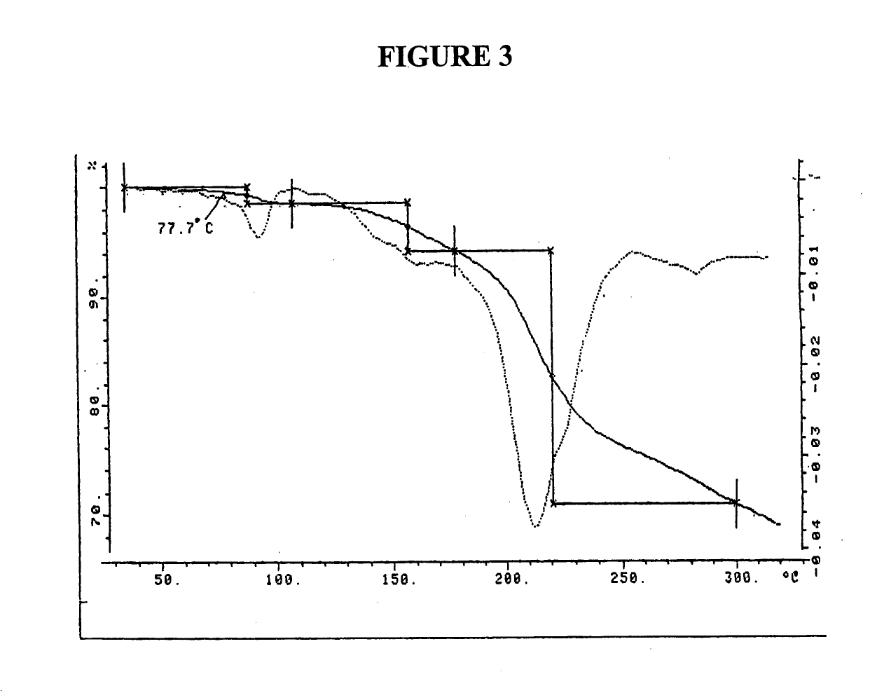

In one embodiment, olmesartan medoxomil substantially in polymorph Form G of the present invention has at least one, and preferably all, of the following properties:
(a) a DSC thermogram with an endothermic peak at about 105.23° C.;
(b) a DSC pattern pattern substantially in accordance with FIG. 1;
(c) a X-ray powder diffraction pattern substantially in accordance with FIG. 2;
(d) a TGA pattern substantially in accordance with FIG. 3; and/or

(e) a XRD pattern comprising characteristic peaks summarized in Table 1 below:

TABLE 1

| Relative | |
|---|---|
| Intensity (%) | Angle (2θ) |
| 28.14 | 4.59006 |
| 24.98 | 8.75645 |
| 21.91 | 9.09096 |
| 48.50 | 10.38614 |
| 28.81 | 10.51912 |
| 58.67 | 13.31240 |
| 88.10 | 13.42330 |
| 15.73 | 14.00358 |
| 18.14 | 15.65376 |
| 27.37 | 16.13896 |
| 24.74 | 16.23844 |
| 62.76 | 16.75931 |
| 60.02 | 17.72515 |
| 40.96 | 18.43222 |
| 22.90 | 19.27729 |
| 20.67 | 19.35142 |
| 30.81 | 20.48076 |
| 26.41 | 21.07227 |
| 25.26 | 21.29568 |
| 21.60 | 21.73946 |
| 100.00 | 23.12553 |
| 18.89 | 23.87661 |
| 21.09 | 24.81594 |
| 25.97 | 28.57259 |
| 42.00 | 28.96676 |

FIG. 3 shows the results of a Thermogavimetric Analysis of polymorph Form G of olmesartan medoxomil. The Thermogavimetric Analysis was performed using a Mettler Toludo TA4000 Series (TG50) thermoanalyzer system. Approximately 8 to 8.5 mg of the sample was accurately weighed in an aluminum crucible. The sample was heated from 35-350° C. @5° C./min under nitrogen pressure.

Yet another aspect of the present invention is directed to pharmaceutical compositions containing at least olmesartan medoxomil substantially in polymorph Form G disclosed herein. Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes. Oral dosage forms include, but are not limited to, tablets, pills, capsules, troches, sachets, suspensions, powders, lozenges, elixirs and the like. The olmesartan medoxomil substantially in polymorph Form G disclosed herein also may be administered as suppositories, ophthalmic ointments and suspensions, and parenteral suspensions, which are administered by other routes. The dosage forms may contain the olmesartan medoxomil substantially in polymorph Form G disclosed herein as is or, alternatively, may contain the olmesartan medoxomil substantially in polymorph Form G disclosed herein as part of a composition. The pharmaceutical compositions may further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

Capsule dosages will contain olmesartan medoxomil substantially in polymorph Form G within a capsule which may be coated with gelatin. Tablets and powders may also be coated with an enteric coating. The enteric-coated powder forms may have coatings comprising phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, they may be employed with suitable plasticizers and/or extending agents. A coated capsule may have a coating on the surface of the capsule or may be a capsule comprising a powder or granules with an enteric-coating.

A composition for tableting or capsule filing can be prepared by wet granulation. In wet granulation, some or all of the active ingredients and excipients in powder form are blended and then further mixed in the presence of a liquid, typically water, which causes the powders to clump up into granules. The granulate is screened and/or milled, dried and then screened and/or milled to the desired particle size. The granulate can then be tableted or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can be compressed subsequently into a tablet. As an alternative to dry granulation, a blended composition can be compressed directly into a compacted dosage form using direct compression techniques. Direct compression produces a more uniform tablet without granules.

Tableting compositions may have few or many components depending upon the tableting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients contemplated by the present invention include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

In one embodiment, the olmesartan medoxomil substantially in polymorph Form G disclosed herein for use in the pharmaceutical compositions of the present invention can have a D₅₀ and D₉₀ particle size of less than about 400 microns, preferably less than about 200 microns, more preferably less than about 150 microns, still more preferably less than about 50 microns and most preferably less than about 15 microns. The particle sizes can be obtained by, for example, any milling, grinding, micronizing or other particle size reduction method known in the art to bring the solid state olmesartan medoxomil substantially in polymorph Form G into any of the foregoing desired particle size range.

Actual dosage levels of olmesartan medoxomil substantially in polymorph Form G may be varied to obtain an amount of olmesartan medoxomil substantially in polymorph Form G that is effective to obtain a desired therapeutic response for a particular composition and method of administration for treatment of a mammal. The selected dosage level therefore depends upon such factors as, for example, the desired therapeutic effect, the route of administration, the desired duration of treatment, and other factors. The total daily dose of the novel polymorph administered to a host in single or divided dose and can vary widely depending upon a variety of factors including, for example, the body weight, general health, sex, diet, time and route of administration, rates of absorption and excretion, combination with other drugs, the severity of the particular condition being treated, etc.

The following example is provided to enable one skilled in the art to practice the invention and is merely illustrative of the present invention. The example should not be read as limiting the scope of the invention as defined in the features and advantages.

### EXAMPLE 1

Preparation of Polymorph Form G of Olmesartan Medoxomil

A four-neck 500 ml flask equipped with a mechanical stirring condenser and thermometer was charged with methanol (100 ml) and olmesartan medoxomil (10 g; 18 mmol) was slowly added. The suspension was slowly heated to a temperature ranging from about 45° C. to about 50° C. and maintained for about 60 minutes. Next, about 60-70 ml of methanol was distilled. The mixture was then slowly cooled to room temperature and then further cooled to a temperature ranging from about 0° C. to about 5° C. such that crystalline olmesartan medoxomil precipitated out of solution. The precipitate product was filtered on a Buchner funnel and washed with methanol (10 ml). The filtered product was then dried to provide polymorph Form G of olmesartan medoxomil (5 g). Yield - 50%.

The average values of diffraction angles and the relative intensities in the powder X-ray diffraction spectrum of olmesartan medoxomil substantially in polymorph Form G are given above in Table 1. The DSC, XRD and TGA of the final product are set forth in FIGS. 1-3, respectively, and were recorded and identified as polymorph Form G of olmesartan medoxomil.

While the above description contains many specifics, these specifics should not be construed as limitations of the invention, but merely as exemplifications of preferred embodiments thereof. Those skilled in the art will envision many other embodiments within the scope and spirit of the invention as defined by the features and advantages appended hereto.

What is claimed is:

1. A process for the preparation of olmesartan medoxomil substantially in polymorph Form G, the process comprising:
(a) preparing a solution comprising olmesartan medoxomil and one or more solvents selected from the group consisting of a nitrile, alcohol and mixtures thereof at a suitable temperature; and
(b) recovering olmesartan medoxomil substantially in polymorph Form G from the solution.

2. The process of claim 1, wherein the solvent is a nitrile selected from the group consisting of acetonitrile, propionitrile and mixtures thereof.

3. The process of claim 1, wherein the solvent is an alcohol having from 1 to about 12 carbon atoms.

4. The process of claim 1, wherein the solvent is an alcohol selected from the group consisting of methanol, ethanol, isopropyl alcohol, n-butyl alcohol, t-butyl alcohol and mixtures thereof.

5. The process of claim 1, wherein the solvent is a nitrile selected from the group consisting of acetonitrile, propionitrile and mixtures thereof and an alcohol selected from the group consisting of methanol, ethanol, isopropyl alcohol, n-butyl alcohol, t-butyl alcohol and mixtures thereof.

6. The process of claim 1, wherein the solvent is present in an amount of about 2 to about 30 volumes.

7. The process of claim 1, wherein the solution is cooled to precipitate olmesartan medoxomil substantially in polymorph Form G out of the solution and the precipitate is filtered to recover olmesartan medoxomil substantially in polymorph Form G.

8. The process of claim 1, wherein the olmesartan medoxomil substantially in polymorph Form G is recovered by crystallization out of the solution.

9. Olmesartan medoxomil substantially in polymorph Form G prepared by the process of claim 1.

10. Olmesartan medoxomil substantially in polymorph Form G.

11. Olmesartan medoxomil substantially in polymorph Form G and exhibiting a characteristic peak (expressed in degrees 2θ±0.2°θ) at about 13.4 and/or 23.1.

12. The olmesartan medoxomil substantially in polymorph Form G of claim 11, further characterized by exhibiting characteristic peaks (expressed in degrees 2θ±0.2°θ) at approximately one or more of the positions: about 13.4, 16.8, 17.7 and/or 23.1.

13. The olmesartan medoxomil substantially in polymorph Form G of claim 10, further characterized by having at least one of the following properties: (a) a differential scanning calorimetric (DSC) thermogram substantially in accordance with FIG. 1, (b) a X-ray diffraction (XRD) pattern substantially in accordance with FIG. 2, and/or (c) a Thermogavimetric Analysis (TGA) substantially in accordance with FIG. 3.

14. The olmesartan medoxomil substantially in polymorph Form G of claim 10, further characterized by having a XRD pattern substantially in accordance with FIG. 2.

15. The olmesartan medoxomil substantially in polymorph Form G of claim 10, further characterized by a DSC thermogram with an endothermic peak at about 105.23° C.

16. A pharmaceutical composition comprising a therapeutically effective amount of olmesartan medoxomil substantially in polymorph Form G and one or more pharmaceutically acceptable excipients.

17. The pharmaceutical composition of claim 16, wherein the olmesartan medoxomil substantially in polymorph Form G is a micronized olmesartan medoxomil substantially in polymorph Form G having a particle size of less than about 400 microns.

18. The pharmaceutical composition of claim 16, wherein the olmesartan medoxomil substantially in polymorph Form G is a micronized olmesartan medoxomil substantially in polymorph Form G having a particle size of less than about 200 microns.

19. The pharmaceutical composition of claim 16, wherein the olmesartan medoxomil substantially in polymorph Form G is a micronized olmesartan medoxomil substantially in polymorph Form G having a particle size of less than about 150 microns.

20. The pharmaceutical composition of claim 16, wherein the olmesartan medoxomil substantially in polymorph Form G is a micronized olmesartan medoxomil substantially in polymorph Form G having a particle size of less than about 50 microns.

21. The pharmaceutical composition of claim 16, wherein the olmesartan medoxomil substantially in polymorph Form G is a micronized olmesartan medoxomil substantially in polymorph Form G having a particle size of less than about 15 microns.

22. The pharmaceutical composition of claim 16, wherein the olmesartan medoxomil substantially in polymorph Form G exhibits a characteristic peak (expressed in degrees 2θ±0.2°θ) at about 13.4 and/or 23.1.

23. The pharmaceutical composition of claim 16, wherein the olmesartan medoxomil substantially in polymorph Form G is further characterized by exhibiting characteristic peaks (expressed in degrees 2θ±0.2°θ) at approximately one or more of the positions: about 13.4, 16.8, 17.7 and/or 23.1.

24. The pharmaceutical composition of claim 16, wherein the olmesartan medoxomil substantially in polymorph Form G is further characterized by having at least one of the following properties: (a) a DSC thermogram substantially in accordance with FIG. 1, (b) a XRD pattern substantially in accordance with FIG. 2, and/or (c) a TGA substantially in accordance with FIG. 3.

25. The pharmaceutical composition of claim 16, wherein the olmesartan medoxomil substantially in polymorph Form G is further characterized by having a XRD pattern substantially in accordance with FIG. 2.

26. The pharmaceutical composition of claim 16, wherein the olmesartan medoxomil substantially in polymorph Form G is further characterized by a DSC thermogram with an endothermic peak at about 105.23° C.

27. A method for treating or preventing hypertension comprising administering to a subject in need of such treatment or prevention a therapeutically effective amount of the olmesartan medoxomil substantially in polymorph Form G of claim 10.
